(19) United States
(12) Patent Application Publication
(10) Pub. No.: US 2023/0081751 A1
(43) Pub. Date: Mar. 16, 2023

(54) METHOD FOR DETERMINING ACCURACY OF HEART RATE VARIABILITY

(71) Applicant: Hoffmann-La Roche Inc., Little Falls, NJ (US)

(72) Inventors: Christopher Hughes Chatham, Little Falls, NJ (US); Joerg Felix Hipp, Basel (CH); Lito Kriara, Basel (CH); Florian Lipsmeier, Basel (CH); Mattia Zanon, Basel (CH)

(73) Assignee: Hoffmann-La Roche Inc., Little Falls, NJ (US)

(21) Appl. No.: 17/795,617

(22) PCT Filed: Feb. 10, 2021
(86) PCT No.: PCT/EP2021/053164

§ 371 Date: Jul. 27, 2022

(30) Foreign Application Priority Data
Feb. 11, 2020 (EP) 20156709.6

(51) Int. Cl. A61B 5/00; A61B 5/024; A61B 5/0245; A61B 5/28

(52) U.S. Cl. CPC A61B 5/7221; A61B 5/02438; A61B 5/02416; A61B 5/02405; A61B 5/7267; A61B 5/0245; A61B 5/7246; A61B 5/28; A61B 2560/0431; A61B 5/352

(57) ABSTRACT

A computer implemented method for determining accuracy of heart rate variability is proposed. The method comprises the following steps:
a) providing at least one photoplethysmogram obtained by at least one portable photoplethysmogram device (110);
b) Determining at least one signal feature by evaluating the photoplethysmogram;
c) Determining the accuracy of heart rate variability by using at least one trained model, wherein the signal features determined in step b) are used as input for the trained model.

### TECHNICAL FIELD

The present invention refers to a method for determining accuracy of heart rate variability. The invention further relates to a portable photoplethysmogram device and to a computer program and a computer-readable storage medium for performing the method according to the present invention. The method and devices, in particular, may be used in the field of wrist-worn devices. Other fields of application of the present invention, however, are feasible.

### BACKGROUND ART

Heart Rate Variability (HRV) is a measure of the time differences between consecutive heart beats primarily caused by the combination of processes controlling cardiac activity. Heart Rate (HR) pacing is regulated by the continuous balance between the sympathetic and parasympathetic branches of the autonomous nervous system, see McCorry, Laurie Kelly. “Physiology of the autonomic nervous system.” American journal of pharmaceutical education 71.4 (2007): 78. The Sympathetic Nervous System (SNS) decreases HRV and is associated with emotional arousal, stressful situations and is responsible for the so called “fight-or-flight” response. The Parasympathetic Nervous System (PNS), on the other hand, increases HRV and governs the “rest and digest” functions when the body is at rest and relaxed. Thus, measuring HRV is a convenient, non-invasive proxy for monitoring variations in the balance between the SNS and PNS in response to endogenous (psychophysiological, behavioral) and exogenous (environmental) stimuli, see Acharya, U. Rajendra, et al. “Heart rate variability: a review.” Medical and biological engineering and computing 44.12 (2006): 1031-1051.

For this reason, HRV is considered a physiological parameter of high interest and it has been used in a wide range of different studies, for example to understand the relation with other relevant physiological variables like blood pressure, see Rivera, Ana Leonor, et al. “Heart rate and systolic blood pressure variability in the time domain in patients with recent and long-standing diabetes mellitus.” PloS one 11.2 (2016): e0148378 and De Boer, R. W., J. M. Karemaker, and J. Strackee. “Relationships between short-term blood-pressure fluctuations and heart-rate variability in resting subjects I: a spectral analysis approach.” Medical and biological engineering and computing 23.4 (1985): 352-358, or its correlation with demographic information like age and gender, see Umetani, Ken, et al. “Twenty-four hour time domain heart rate variability and heart rate: relations to age and gender over nine decades.” Journal of the American College of Cardiology 31.3 (1998): 593-601 and Zhang, John. “Effect of age and sex on heart rate variability in healthy subjects.” Journal of manipulative and physiological therapeutics 30.5 (2007): 374-379, in heart diseases, see Thayer, Julian F., Shelby S. Yamamoto, and Jos F. Brosschot. “The relationship of autonomic imbalance, heart rate variability and cardiovascular disease risk factors.” International journal of cardiology 141.2 (2010): 122-131, Chen, Wenhui, et al. “A CHF detection method based on deep learning with RR intervals.” 2017 39th Annual International Conference of the IEEE Engineering in Medicine and Biology Society (EMBC). IEEE, 2017 and Reed, Matt J., C. E. Robertson, and P. S. Addison. “Heart rate variability measurements and the prediction of ventricular arrhythmias.” Qjm 98.2 (2005): 87-95, and diabetes, see Malpas, Simon C., and Timothy J B Maling. “Heart-rate variability and cardiac autonomic function in diabetes.” Diabetes 39.10 (1990): 1177-1181, sleep quality, see Tobaldini, Eleonora, et al. “Heart rate variability in normal and pathological sleep.” Frontiers in physiology 4 (2013): 294 and Trinder, John, et al. “Autonomic activity during human sleep as a function of time and sleep stage.” Journal of sleep research 10.4 (2001): 253-264, or considered as a biomarker in drugs understanding, see Silke, B., C. Campbell, and D. King. “The potential cardiotoxicity of antipsychotic drugs as assessed by heart rate variability.” Journal of psychopharmacology 16.4 (2002): 355-360 and Lotufo, Paulo A., et al. “A systematic review and meta-analysis of heart rate variability in epilepsy and antiepileptic drugs.” Epilepsia 53.2 (2012): 272-282, and to measure cardiovascular fitness, see Buchheit, Martin, and Cyrille Gindre. “Cardiac parasympathetic regulation: respective associations with cardiorespiratory fitness and training load.” American Journal of Physiology-Heart and Circulatory Physiology 291.1 (2006): H451-H458 and De Meersman, Ronald Edmond. “Heart rate variability and aerobic fitness.” American heart journal 125.3 (1993): 726-731, to mention just a few.

Historically, the Electrocardiogram (ECG) signal has been the standard signal provider of consecutive intervals given the distinctive shape of the QRS complex that makes it a convenient fiducial point for identifying single heartbeats from which R-to-R Intervals (RRIs). These technique, however, has the drawback that change of HR is only available during testing with the ECG but not for daily life activities.

In the last decade, Photoplethysmogram (PPG) wrist-worn devices became more common in the consumer field, after being a very important tool in clinical settings given their ability to provide medical grade vital signs such as blood oxygenation and pulse rate. Consumer PPG devices comprise a LED emitting light into the skin and a photodiode for measuring the reflected photons. The reflected light shows a pulsatile component caused by blood volume variations in the skin and underlying tissues due to the heart beat, making the PPG waveform signal a good candidate for identifying surrogate RRIs for calculating HRV. Wearable consumer devices are also comfortable to wear during daily life activities, showing the potential to provide frequent measurements in uncontrolled conditions outside the clinic. However, the reliability of the vital signs provided by PPG consumer wearables is hindered by several cofounders, among them blood perfusion and motion artefacts, see Elgendi, Mohamed. “On the analysis of fingertip photoplethysmogram signals.” Current cardiology reviews 8.1 (2012): 14-25, lowering the signal quality and thus the confidence with which vital signs are estimated. In this context, early work has been done to define and develop PPG waveform quality metrics, e.g. by looking only at the presence of artifacts in the signals, see Robles-Rubio, Carlos A., Karen A. Brown, and Robert E. Kearney. “A new movement artifact detector for photoplethysmographic signals.” 2013 35th Annual International Conference of the IEEE Engineering in Medicine and Biology Society (EMBC). IEEE, 2013 and Karlen, Walter, et al. “Photoplethysmogram signal quality estimation using repeated Gaussian filters and cross-correlation.” Physiological measurement 33.10 (2012): 1617, or by manually annotating the PPG waveform and use features of the signal to build a supervised classifier, see Sukor, J. Abdul, S. J. Redmond, and N. H. Lovell. “Signal quality measures for pulse oximetry through waveform morphology analysis.” Physiological measurement 32.3 (2011): 369, Li, Qiao, and Gari D. Clifford. “Dynamic time warping and machine learning for signal quality assessment of pulsatile signals.” Physiological measurement 33.9 (2012): 1491, Elgendi, Mohamed. “Optimal signal quality index for photoplethysmogram signals.” Bioengineering 3.4 (2016): 21 and Orphanidou, Christina, et al. “Signal-quality indices for the electrocardiogram and photoplethysmogram: Derivation and applications to wireless monitoring.” IEEE journal of biomedical and health informatics 19.3 (2014): 832-838.

US 2019/110755 A1 describes a model of data quality which is derived for physiological monitoring with a wearable device by comparing data from the wearable device to concurrent data acquisition from a ground truth device such as a chest strap or electrocardiography (EKG) heart rate monitor. With this comparative data, a machine learning model or the like may be derived to prospectively evaluate data quality based on the data acquisition context, as determined, for example, by other sensor data and signals from the wearable device.

Yoshida Seiya et al.: “A Heartbeat Interval Error Compensation Method Using Multiple Linear Regression for Photoplethysmography Sensors”, 2019 IEEE BIO-MEDICAL CIRCUITS AND SYSTEMS CONFERENCE (BIOCAS), IEEE, 17 Oct. 2019, pages 1-4, XP033644565, DOI: 10.1109/BIOCAS.2019.8918719 describes an error compensation method for heartbeat intervals measured by a photoplethysmography (PPG) sensor. US 2018/249964 A1 describes deep learning algorithms for heartbeats detection.

Despite the advantages of such known methods, using manual annotations implies the risk of mistakes due to mislabeling. Moreover, distinguishing between trustworthy and untrustworthy HRV data points is not possible.

### Problem to be Solved

It is therefore desirable to provide methods and devices which address the above-mentioned technical challenges of determining heart rate variability and its quality. Specifically, methods and devices shall be proposed which overcome the need for manual annotations.

### SUMMARY

This problem is addressed by a method and a portable photoplethysmogram device for determining accuracy of heart rate variability with the features of the independent claims. Advantageous embodiments which might be realized in an isolated fashion or in any arbitrary combinations are listed in the dependent claims.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the invention, a computer implemented method for determining accuracy of heart rate variability is disclosed.

The term “computer implemented method” as used herein is a broad term and is to be given its ordinary and customary meaning to a person of ordinary skill in the art and is not to be limited to a special or customized meaning. The term specifically may refer, without limitation, to a method involving at least one computer and/or at least one computer network. The computer and/or computer network may comprise at least one processor which is configured for performing at least one of the method steps of the method according to the present invention. Preferably each of the method steps is performed by the computer and/or computer network. The method may be performed completely automatically, specifically without user interaction. The term “automatically” as used herein is a broad term and is to be given its ordinary and customary meaning to a person of ordinary skill in the art and is not to be limited to a special or customized meaning. The term specifically may refer, without limitation, to a process which is performed completely by means of at least one computer and/or computer network and/or machine, in particular without manual action and/or interaction with a user.

The method comprises the following steps which, as an example, may be performed in the given order. It shall be noted, however, that a different order is also possible. Further, it is also possible to perform one or more of the method steps once or repeatedly. Further, it is possible to perform two or more of the method steps simultaneously or in a timely overlap-ping fashion. The method may comprise further method steps which are not listed.

The method comprises the following steps:
a) providing at least one photoplethysmogram obtained by at least one portable photoplethysmogram device;
b) Determining at least one signal feature by evaluating the photoplethysmogram;
c) Determining the accuracy of heart rate variability by using at least one trained model, wherein the signal features determined in step b) are used as input for the trained model.

The term “heart rate variability” (HRV) as used herein is a broad term and is to be given its ordinary and customary meaning to a person of ordinary skill in the art and is not to be limited to a special or customized meaning. The term specifically may refer, without limitation, to a measure of regularity between consecutive heartbeats.

The term “plethysmogram” as used herein is a broad term and is to be given its ordinary and customary meaning to a person of ordinary skill in the art and is not to be limited to a special or customized meaning. The term specifically may refer, without limitation, to is a result of a measurement of volume changes of at least one part of the human body or of organs. The term “photoplethysmogram” (PPG) as used herein is a broad term and is to be given its ordinary and customary meaning to a person of ordinary skill in the art and is not to be limited to a special or customized meaning. The term specifically may refer, without limitation, to an optically determined plethysmogram. The PPG may show development of a signal from the PPG device over time.

The PPG may comprise a plurality of beats. The term “beat” of the PPG as used herein is a broad term and is to be given its ordinary and customary meaning to a person of ordinary skill in the art and is not to be limited to a special or customized meaning. The term specifically may refer, without limitation, to at least one local maximum of the PPG. The heart rate variability may be measured by the variation in the beat-to-beat intervals, also denoted R-to-R intervals (RRI). Generally, an R wave is a section of an ECG signal consisting of a sharp raise followed by a sharp decrease of the signal. The morphology of a PPG signal may be different from the ECG one but still showing repetitive pattern due to heart beats. The heart rate variability may be defined as the variation of successive heartbeats.

The term “accuracy” as used herein is a broad term and is to be given its ordinary and customary meaning to a person of ordinary skill in the art and is not to be limited to a special or customized meaning. The term specifically may refer, without limitation, to is a measure for closeness of a measurement value to a certain value, in particular a true value. The true value may be a heart rate variability value determined using at least one Electrocardiogram (ECG) device.

The term “providing” as used herein is a broad term and is to be given its ordinary and customary meaning to a person of ordinary skill in the art and is not to be limited to a special or customized meaning. The term specifically may refer, without limitation, to a process of determining and/or generating and/or making available the photoplethysmogram.

The term “photoplethysmogram device” as used herein is a broad term and is to be given its ordinary and customary meaning to a person of ordinary skill in the art and is not to be limited to a special or customized meaning. The term specifically may refer, without limitation, to at least one device configured for determining at least one PPG.

The photoplethysmogram device may comprise at least one illumination source. The term “illumination source” as used herein is a broad term and is to be given its ordinary and customary meaning to a person of ordinary skill in the art and is not to be limited to a special or customized meaning.

The term specifically may refer, without limitation, to at least one arbitrary device configured for generating at least one light beam. The illumination source may comprise at least one light source such as at least one light-emitting-diode (LED) transmitter. The illumination source may be configured for generating at least one light beam for illuminating e.g. the skin on at least one part of the human body. The illumination source may be configured for generating light in the red, infrared or green spectral region. As used herein, the term “light”, generally, refers to a partition of electromagnetic radiation which is, usually, referred to as “optical spectral range” and which comprises one or more of the visible spectral range, the ultraviolet spectral range and the infrared spectral range. Herein, the term “ultraviolet spectral range”, generally, refers to electromagnetic radiation having a wavelength of 1 nm to 380 nm, preferably of 100 nm to 380 nm. The term “visible spectral range”, generally, refers to a spectral range of 380 nm to 760 nm. The term “infrared spectral range” (IR) generally refers to electromagnetic radiation of 760 nm to 1000 μm, wherein the range of 760 nm to 1.5 μm is usually denominated as “near infrared spectral range” (NIR) while the range from 1.5μ to 15 μm is denoted as “mid infrared spectral range” (MidIR) and the range from 15 μm to 1000 μm as “far infrared spectral range” (FIR).

The photoplethysmogram device may comprise at least one photodetector, in particular at least one photosensitive diode. The term “photodetector” as used herein is a broad term and is to be given its ordinary and customary meaning to a person of ordinary skill in the art and is not to be limited to a special or customized meaning. The term specifically may refer, without limitation, to at least one light-sensitive device for detecting a light beam, such as for detecting an illumination generated by at least one light beam. The photodetector may be configured for detecting light from transmissive absorption and/or reflection in response to illumination by the light generated by the illumination source.

The term “signal” of the PPG device as used herein is a broad term and is to be given its ordinary and customary meaning to a person of ordinary skill in the art and is not to be limited to a special or customized meaning. The term specifically may refer, without limitation, to at least one electronic signal of the PPG device, in particular of the photodetector, depending on detected light from transmissive absorption and/or reflection in response to illumination by the light generated by the illumination source.

The term “portable” as used herein is a broad term and is to be given its ordinary and customary meaning to a person of ordinary skill in the art and is not to be limited to a special or customized meaning. The term specifically may refer, without limitation, to property of the PPG device allowing that a user can hold and/or wear and/or transport the PPG device. Specifically, the portable PPG device may be wearable. For example, the PPG device may be a wristwatch such as a smartwatch. Using a portable PPG device may result in that disturbances can influence the HRV measurement such as motions artefacts. Uncontrolled conditions met in daily life may pose several challenges related to disturbances that can deteriorate the PPG signal making the calculation of the HRV untrustworthly and not reliable.

The term “evaluating the photoplethysmogram” as used herein is a broad term and is to be given its ordinary and customary meaning to a person of ordinary skill in the art and is not to be limited to a special or customized meaning. The term specifically may refer, without limitation, to analysis of the PPG such as using at least one filtering technique. The photoplethysmogram may comprises at least one signal, also denoted as PPG signal. The method may comprise evaluating the signal. The evaluation may comprise one or more of interpolating the signal, resampling the signal, isolating signal components, analyzing considering non-overlapping windows, normalizing, identifying of peaks.

For example, the PPG signal may be interpolated over a uniform time grid to account for slight fluctuations of sampling frequency, such as around 20 Hz. The PPG signal may be resampled to increase the sampling frequency, such as to 1 kHz, for example by using an averaging filter of length 0.5 seconds and a Blackman window.

The PPG signal may comprise a slow trend, often referred to DC component. Without being bound by this theory, this trend is likely due to respiration and other low frequency physiology-related modulations, see Julien, Claude. “The enigma of Mayer waves: facts and models.” Cardiovascular research 70.1 (2006): 12-21. The PPG signal may comprise a pulsatile component, often referred to AC, due to blood volume variations synchronized with the heart beats. To isolate the AC component from the PPG signal a Morlet wavelet may be used, i.e. a very selective band pass filter, centered around the frequency of interest, i.e. heart rate. For additional details about the Morlet wavelet reference is made toCohen, Michael X. “A better way to define and describe Morlet wavelets for time-frequency analysis.” NeuroImage 199 (2019): 81-86. It was found that the accuracy values are the key to make sure that the filtered signal contains the pulsatile component and not, for example, motion artefacts.

The PPG signal, in particular the resampled and interpolated PPG signal, may be analysed considering non-overlapping windows, such as windows of 20 seconds. The term “window”, also denoted time window, as used herein is a broad term and is to be given its ordinary and customary meaning to a person of ordinary skill in the art and is not to be limited to a special or customized meaning. The term specifically may refer, without limitation, to a time span. For each window a median heart rate may be determined. The method may comprise using the median heart rate to build wavelet filter coefficients. Before applying the filter, a PPG waveform in a current window may be normalized by a DC mean value.

The peaks on the filtered PPG signal may then be identified and/or determined and/or calculated looking at a combination of first and second derivatives of the signal. Identified peaks may then be concatenated until the last window that has been analyzed. A RRI time series, i.e. a specific number of consecutive peaks, may be filtered with a heuristic rule to make sure erroneous beats are excluded from the calculation of the HRV statistics. For example, a current RRI may be kept when it differs less than 30% from the previous one and the previous one, i.e. differs less than 30% from the one before, i.e. RRIᵢ₋₂. Otherwise the RRI may be removed from the RRI time series.

The term “signal feature” as used herein is a broad term and is to be given its ordinary and customary meaning to a person of ordinary skill in the art and is not to be limited to a special or customized meaning. The term specifically may refer, without limitation, to a feature characterizing behavior of the signal in a time window of interest. The signal features may comprise both statistics describing the PPG signal as well as statistics describing the RRI distributions. The former ones may comprise one or more of variance, minimum, maximum, average, standard deviation, entropy, kurtosis and skewness values over raw and filtered PPG signals. The latter ones may comprise one or more of average RRIs and HR, the absolute number of filtered RRIs and the ratio between good and filtered RRIs, the minimum and maximum number of RRIs as well as the 5th and 95th RRI percentiles. The signal feature may be determined for a current time instant tᵢ considering RRIs temporally located between the current time instant tᵢ and a time instant tᵢ-wl, wherein wl is a window length ranging from seconds, e.g. 30 seconds, to minutes, such as 5 minutes. The signal feature comprise at least one feature selected from the group consisting of: root mean square of successive differences (RMSSD), standard deviation of the RRI intervals (SDNN), standard deviation of the RRIs in a current window, pnn50 from PPG, root mean square of pnn50, average RRI value from PPG in the current window, average heart rate from PPG in the current window, number of ectopic RRIs in the current window, ratio between number of ectopic and normal RRIs in the current window, minimum RRI value in the current window, maximum RRI value in the current window, variance of the RRIs in the current window, number of RRIs in the current window, 95th percentile of the RRIs in the current window, 5th percentile of the RRIs in the current window, variance of a raw, i.e. not filtered, PPG signal in the current window, max value of the raw PPG signal in the current window, min value of the raw PPG signal in the current window, average value of the raw PPG signal in the current window, standard deviation of the raw PPG signal in the current window, entropy of the raw PPG signal in the current window, kurtosis of the raw PPG signal in the current window, skewness of the raw PPG signal in the current window, variance of the filtered PPG signal in the current window, max value of the filtered PPG signal in the current window, min value of the filtered PPG signal in the current window, average value of the filtered PPG signal in the current window, standard deviation of the filtered PPG signal in the current window, kurtosis of the filtered PPG signal in the current window, skewness of the filtered PPG signal in the current window. In step b) all of these signal features may be determined or a subset of these signal features may be determined. It was found that the following subset of features is particular suitable for a reliable determination of accuracy of heart rate variability: root mean square of successive differences (RMSSD), standard deviation of the RRI intervals (SDNN), standard deviation of the RRIs in a current window, pnn50 from PPG, average heart rate from PPG in the current window, number of ectopic RRIs in the current window, minimum RRI value in the current window, variance of the RRIs in the current window, number of RRIs in the current window, 95^{th} percentile of the RRIs in the current window. The RMSSD may be determined by calculating the square root of the mean of the squares of the successive differences of consecutive RRIs:

$$RMSSD = \sqrt{\frac{{{Σ}_{i = 0}^{N - 1} ( {RRI}_{i} - {RRI}_{i + 1} )}^{2}}{N - 1}} .$$

The SDNN may be determined by calculating:

$$S D N N = \sqrt{\frac{{{Σ}_{i = 1}^{N} ( {RRI}_{i} - \overline{RRI} )}^{2}}{N - 1}} ,$$

where is RRI the average of the RRI in the considered time window. pnn50 is the proportion of NN50 divided by total number of RRIs, wherein NN50 is the number of pairs of successive RRIs that differ by more than 50 ms.

The term “trained model” as used herein is a broad term and is to be given its ordinary and customary meaning to a person of ordinary skill in the art and is not to be limited to a special or customized meaning. The term specifically may refer, without limitation, to a model for predicting accuracy which was trained on at least one training dataset, also denoted training data. The method may comprise at least one training step, wherein, in the training step, the trained model is trained on the at least one training dataset. The trained model may comprise at least one model selected from the group consisting of: a linear regression model, e.g. comprising transformed features, such as log-transformed or polynomial; at least one non-linear Artificial Neural Network (ANN), in particular at least one deep learning architecture such as Convolutional NN, Recurrent NN, Long Short Term Memory NN, and the like; at least one Support Vector Machine (SVM); at least one kernel based method; Tree regression; Random Forest.

The training dataset may comprise of a set of HRV values determined by using the ECG device and HRV values determined by using the PPG device. ECG and PPG data may be collected simultaneously. The training dataset may be determined by performing at least one test protocol comprising a series of activities. The protocol may comprise of a series of activities meant to induce HRV variations so to compare HRV over a wide range of values as well as inducing motion artefacts to test the ability of the algorithm and the quality metric to distinguish between accurate and inaccurate HRV values. Some protocol activities, e.g. console gaming, mental stress manipulation and physical activity, may be included to reflect typical activities performed in daily life use of the PPG device. Pace breathing may be considered because it increases the range of HRV values through respiratory sinus arrhythmia, allowing the calculation of results over a broad range of variation and making easier the post alignment/synchronization of the time series obtained from the reference ECG and the PPG signals. The following table gives a list of an exemplary protocol:

| Activity | Duration |
|---|---|
| Screening & Informed consent process (while sitting, | — |
| at rest) | |
| Placement of ECG and PPG sensors (while sitting, | — |
| at rest) | |
| Baseline (sitting, at rest) | 5 minutes |
| Paced breathing (ladder of increasing respiratory | 5 minutes |
| frequencies from 5 to 20 breaths per minute with | |
| steps of 5) | |
| Console gameplay (PS4 Aaero) | 5 minutes |
| Orthostasis (standing, otherwise at rest) | 5 minutes |
| Mental stress manipulation (Serial 7s [subtraction | 5 minutes |
| by 7 from 700, with eyes closed, pronouncing aloud | |
| each response]) | |
| Physical activity manipulation (uninterrupted indoor | 5 minutes |
| walking along a pre-set circular path; same path for | |
| all subjects) | |
| Baseline (sitting, at rest) | 5 minutes |
| Retrieve PPG/ECG equipment and debrief | — |

The method may comprise analyzing ECG and PPG data to obtain the RRIs time series from which heart rate variability metrics can be derived. The method may comprise comparing the heart rate variability metrics against each other to obtain a measure of the accuracy.

The PPG signals may be evaluated as described above. The signal features from the PPG signal and from the ECG signal may be calculated over the same time window.

For the ECG data comprising a plurality of ECG signals, a different evaluation may be performed. The raw ECG signal may be analyzed with a variation on the Pan-Tompkins algorithms, see Pan J, Tompkins W J. A real-time QRS detection algorithm. IEEE Trans Biomed Eng. 1985 March; 32(3):2. A Savitzky-Golay differentiation filter may be used to provide a filtered version of the raw signal first derivative, see Savitzky, A., Golay, M. J. E. “Smoothing and Differentiation of Data by Simplified Least Squares Procedures”. Analytical Chemistry. 1964, 36(8): 1627-39.doi:10.1021/ac60214a047. The ECG signal may be squared for emphasizing higher frequencies and filtered with a moving integrator filter, e.g. of width 60 ms, i.e. the average QRS complex width, for obtaining the ECG shape back with highlighted QRS complexes. The signal may be normalized with its envelope that is obtained at each time instant by filtering the root mean square of the signal in a rolling window of length Fs/2 with a Butterworth low pass filter with cutoff frequency at, e.g. 0.8 Hz, where Fs represents the sampling frequency of the ECG signal. Single heart beats may be identified on this normalized signal as the peaks exceeding a threshold that in our case was identified as the 90th percentile of the data in the current window. Each heart beat crossing the threshold may be subsequently checked manually to make sure no erroneous beat was included in the analysis.

The method may comprise determining at least one heart rate variability metric of the heart rate variability values determined by using at least one electrocardiogram device. The method may comprise determining at least one heart rate variability metric of the heart rate variability values determined by using the PPG device. The term “metric” as used herein is a broad term and is to be given its ordinary and customary meaning to a person of ordinary skill in the art and is not to be limited to a special or customized meaning. The term specifically may refer, without limitation, to an indicator expressing in a number a certain quantity. The term “heart rate variability metric” as used herein is a broad term and is to be given its ordinary and customary meaning to a person of ordinary skill in the art and is not to be limited to a special or customized meaning. The term specifically may refer, without limitation, to statistics calculated on RRIs contained inside a time window of specific length that can last from minutes to hours. Specifically, a HRV metric is a number expressing the vari-ability between heartbeats. For further details about the heart rate variability metric reference is made to Fei, Lu, et al, “Short- and long-term assessment of heart rate variability for risk stratification after acute myocardial infarction.” The American journal of cardiology 77.9 (1996): 681-684 and Mourot, Laurent, et al. “Short- and long-term effects of a single bout of exercise on heart rate variability: comparison between constant and interval training exercises.” European journal of applied physiology 92.4-5 (2004): 508-517. In particular, the heart rate variability metrics may be calculated on time window of minutes, what in the literature is sometimes referred as “short-term” heart rate variability. The heart rate variability metrics may be calculated in the specific 30, 60, 90, 120, 180, 240 and 300 seconds. Heart rate variability metrics may belong to different classes depending on the domain of the method used to analyze the RRIs.

For example, the heart rate variability metrics may comprise the time, frequency, non-linear and geometrical domains. The heart rate variability metrics may comprise the Root Mean of the Squared Differences (RMSSD) of consecutive RRIs:

$$RMSSD = \sqrt{\frac{{{Σ}_{i = 0}^{N - 1} ( {RRI}_{i} - {RRI}_{i + 1} )}^{2}}{N - 1}}$$

and the Standard Deviation of the NN intervals (SDNN):

$$SDNN = \sqrt{\frac{{{Σ}_{i = 1}^{N} ( {RRI}_{i} - \overline{RRI} )}^{2}}{N - 1}}$$

where is RRI the average of the RRI in the considered time window. Another metric derived from the interval differences may comprise the PNN50, that is, the number of consecutive RRIs differing more than 50 ms normalized by the total number of RRIs in the considered window.

The heart rate variability metrics obtained from the PPG and the ECG may be combined to define a heart rate variability error, also denoted error of heart rate variability. The method may comprise determining at least one error of heart rate variability, i.e. the difference between heart rate variability values obtained with the PPG and the ECG. Specifically, for each heart rate variability metric an error may be determined, at each time instant i-th, as the absolute difference between the heart rate variability values obtained from the PPG and ECG signals. As an example, the error at the time instant i-th for the SDNN metric may be defined as

Err_{SDNN,i}=|SDNN_{ECG,i}−SDNN_{PPG,i}|.

The method may comprise considering a combination of heart rate variability error metrics where at each time instant i-th, the multivariate error metric Err_{multivariate,i} is the average of the errors Err_{SDNN.i} for each heart rate variability metric.

The method may comprise determining a multivariate error metric based on a combination of several HRV metrics errors.

The error of heart rate variability may be used together with signal features extracted from the PPG for determining the trained model for determining the heart rate variability accuracy itself. The term “determining the trained model” as used herein is a broad term and is to be given its ordinary and customary meaning to a person of ordinary skill in the art and is not to be limited to a special or customized meaning. The term specifically may refer, without limitation, to determining coefficients of the model. The method may comprise performing at least one multivariate supervised regression, wherein as input the at least one signal feature extracted from the photoplethysmogram may be used. The output may be the error between the heart rate variability metrics obtained from the PPG signal and the ones obtained from the ECG signal.

The determining of the heart rate variability accuracy may comprise predicting the accuracy based on the actual PPG signal. The trained model can be used to estimate the heart rate variability error (HRVE), e.g. prospectively, when ECG data is not available.

For example, as model a linear model in the form may be used:

HRVE=Xβ

where HRVE is a (n×1) vector collecting the HRVE values Err_{multivriate.i}, X is the (n×p) matrix collecting the features obtained from the PPG and β is the (p×1) vector collecting the model coefficients. The i-th row of matrix X collects the p features calculated in the same time window of PPG data that is used to calculate the i-th heart rate variability value. For example, as estimation technique a Least Absolute Shrinkage and Selection Operator (LASSO) may be used. These techniques may comprise a L1 norm regularization and has the property of setting to zero coefficients in the model associated with unimportant features, allowing to control for complexity and avoiding overfitting, see Tibshirani R. Regression Shrinkage and Selection via the lasso. Journal of the Royal Statistical Society. Series B (methodological). 1996 58(1): 267-88.

In one embodiment the HRV accuracy model may be trained with a subset of signal features:

$$H R {V}_{ACCURACY} ( {t}_{i} ) = {β}_{1} r m s s {d}_{p p g} ( {t}_{i} ) + {β}_{2} p n n 5 {0}_{p p g} ( {t}_{i} ) + {β}_{3} {avg\_hr}_{P P G} ( {t}_{i} ) + {β}_{4} n\_ectpc {\_rri}_{p p g} ( {t}_{i} ) + {β}_{5} min\_rri \_ppg ( {t}_{i} ) + + {β}_{6} {var}_{{rri}_{p p g} ( {t}_{i} )} + {β}_{7} {std}_{r r {i}_{p p g} ( {t}_{i} )} + {β}_{8} {n}_{{rri}_{p p g} ( {t}_{i} )} + {β}_{9} 95 {perc}_{r r {i}_{p p g} ( {t}_{i} )} ,$$

wherein βⱼ are the respective model coefficients, rmssdppg is the RMSSD from the PPG, pnn50_{ppg} is the pnn50 from the PPG, avg_hr_{PPG} is the average heart rate from the PPG in the current window, n_ectpc_rri_{ppg} is the number of ectopic RRIs in the current window, min_rri_ppg is the minimum RRI value in the current window, var_rri_ppg is the variance of the RRIs in the current window, std_rri_ppg is the standard deviation of the RRIs in the current window, n_rri_ppg is the number of RRIs in the current window and 95perc_rri_ppg is the 95th percentile of the RRIs in the current window.

The method, in particular the training step, may comprise at least one validation step, wherein a Leave-One-Subject-Out Cross-Validation (LOSO-CV) is used. At each iteration N−1 subjects out of N subjects may be used to train the model. In the validation step, trained model is tested on the data from the subject that was left out from the training dataset, see Friedman, Jerome, Trevor Hastie, and Robert Tibshirani, The elements of statistical learning. Vol. 1. No. 10. New York: Springer series in statistics, 2001.

The accuracy determined in step c) may be used as quality indicator for heart rate variability data. A better accuracy should be associated with a high quality and a low accuracy with a bad quality. The accuracy may be reflected by a quality metric. The heart rate variability determined from the photoplethysmogram may be an actual value in the quality metric. The quality metric may set a tolerance range that defines acceptable data points. The term “acceptable” as used herein is a broad term and is to be given its ordinary and customary meaning to a person of ordinary skill in the art and is not to be limited to a special or customized meaning. The term specifically may refer, without limitation, to trustworthy and/or reliable data points. The quality metric may be used for deciding and/or differentiating and/or distinguish between acceptable and non-acceptable heart rate variability data points.

The method may comprise comparing the accuracy to at least one threshold. If the accuracy is below the threshold, a heart rate variability data point may be considered as acceptable, otherwise as non-acceptable. The threshold may be used to distinguish between acceptable and unacceptable heart rate variability values. The method may comprise a binary decision to include or exclude a heart rate variability data point. The threshold may be a pre-determined or pre-defined threshold. The threshold may be set on the continuous HRVE values estimated by the trained model. When HRVE is below the threshold the respective heart rate variability value or data point may be considered with good quality and as “acceptable”, otherwise it is not and is considered as “non-acceptable”. An acceptable heart rate variability value may have a heart rate variability quality equal to 1 and a non-acceptable heart rate variability value may have a heart rate variability quality equal to 0.

The method may comprise determining the threshold, in particular at least one threshold level. Influences of different threshold levels may be tested as follows. For example, for all the considered heart rate variability metrics, the calculation of the heart rate variability accuracy may be performed using at least one performance metrics as a function of the threshold levels.

For example, a performance metric can be the Mean Absolute Relative Deviation (MARD):

$$M A R D = 100 \sqrt{\frac{1}{N - 1} {\sum }_{i = 0}^{N} \frac{\vert H R {V}_{e c g , i} - H R {V}_{p p g , i} \vert}{H R {V}_{e c g , i}}}$$

or the Root Mean Squared Error (RMSE):

$$RMSE = \sqrt{\frac{1}{N - 1} {\sum }_{i = 0}^{N} {( {HRV}_{e c g , i} - H R {V}_{p p g , i} )}^{2}}$$

where N represents the number of heart rate variability values, may be used. The performance metric may be an indicator of accuracy. To measure the amount of data with good quality, i.e. the number of accurate heart rate variability values, an additional metric may be considered, calculated as the percentage of heart rate variability values with good quality relative to the total amount of heart rate variability values.

Additionally or alternatively, the influences may be tested using an analysis considering errors arising from setting a threshold on a continuous value, HRVE, which is estimated by a model and thus presents uncertainty. The analysis may thus be highly dependent on the ability of the trained model to accurately predict HRVE. For example, a Receiver Operating Characteristic (ROC) analysis may be used using the true and the predicted values of HRVE for different threshold levels. For each threshold value, a confusion matrix may be calculated, a True Positive Rate (TPR), i.e. the rate of good quality HRV values classified as such, may be determined and a False Positive Rate (FPR), i.e. the number of inaccurate HRV values that are nevertheless included in the analysis because of the uncertainty in the predicted HRVE, may be determined. The heart rate variability accuracy of those points identified as FPR may have an indication of heart rate variability accuracy degradation derived from including these points.

The present invention proposes selecting the optimal value of the threshold to set on the model output. This is different compared to the prior art since the threshold is not set before the model.

Moreover, the present invention proposes determining an error measure. The threshold, in particular the threshold value or values, may be selected by maximizing accuracy of HRV.

In a further aspect of the present invention, a portable photoplethysmogram device is disclosed. The wherein the portable photoplethysmogram device is configured for determining accuracy of heart rate variability. The portable photoplethysmogram device comprises at least one illumination source and at least one photodetector configured for determining at least one photoplethysmogram. The portable photoplethysmogram device further comprises at least one processing unit configured for determining at least one signal feature by evaluating the photoplethysmogram. The processing unit is configured for determining the accuracy of heart rate variability by using at least one trained model, wherein the determined signal features are used as input for the trained model.

Specifically, the portable photoplethysmogram device may be configured for performing the method according to the present invention and/or for being used in the method according to the present invention. For definitions of the features of the portable photoplethysmogram device and for optional features of the portable photoplethysmogram device, reference may be made to one or more of the embodiments of the method as disclosed above or as disclosed in further detail below.

The term “processing unit” as generally used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. In particular, the processing unit may be configured for processing basic instructions that drive the computer or system. As an example, the processing unit may comprise at least one arithmetic logic unit (ALU), at least one floating-point unit (FPU), such as a math co-processor or a numeric coprocessor, a plurality of registers, specifically registers configured for supplying operands to the ALU and storing results of operations, and a memory, such as an L1 and L2 cache memory. In particular, the processing unit may be a multi-core processor. Specifically, the processing unit may be or may comprise a central processing unit (CPU). Additionally or alternatively, the processing unit may be or may comprise a microprocessor, thus specifically the processing unit's elements may be contained in one single integrated circuitry (IC) chip. Additionally or alternatively, the processing unit may be or may comprise one or more application-specific integrated circuits (ASICs) and/or one or more field-programmable gate arrays (FPGAs) or the like. The processing unit specifically may be configured, such as by software programming, for performing one or more evaluation operations.

In a further aspect of the present invention, a computer program is disclosed, the computer program comprising instructions which, when the program is executed by the portable photoplethysmogram device according to the present invention, such as according to any one of the embodiments disclosed above and/or according to any one of the embodiments disclosed in further detail below, cause the portable photoplethysmogram device to carry out steps a) to c) of the method according to the present invention, such as according to any one of the embodiments disclosed above and/or according to any one of the embodiments disclosed in further detail below. For the steps which are not computer-implemented or computer-implementable, the computer program may imply a prompting of the user to perform specific acts.

Similarly, a computer-readable storage medium is disclosed, comprising instructions which, when executed by the portable photoplethysmogram device according to the present invention, such as according to any one of the embodiments disclosed above and/or according to any one of the embodiments disclosed in further detail below, cause the portable photoplethysmogram device to carry out steps a) to c) of the method according to the present invention, such as according to any one of the embodiments disclosed above and/or according to any one of the embodiments disclosed in further detail below.

As used herein, the term “computer-readable storage medium” specifically may refer to a non-transitory data storage means, such as a hardware storage medium having stored there-on computer-executable instructions. The computer-readable data carrier or storage medium specifically may be or may comprise a storage medium such as a random-access memory (RAM) and/or a read-only memory (ROM).

The computer program may also be embodied as a computer program product. As used herein, a computer program product may refer to the program as a tradable product. The product may generally exist in an arbitrary format, such as in a paper format, or on a computer-readable data carrier and/or on a computer-readable storage medium. Specifically, the computer program product may be distributed over a data network.

The methods and devices according to the present invention may provide a large number of advantages over similar methods and devices known in the art. Specifically, the method and devices propose a different approach in view of manual annotations for determining acceptable heart rate variability data points. The method and devices propose to associate to each estimated heart rate variability value a quality metric reflecting its accuracy. In particular, the definition of the quality indicator is made directly on the heart rate variability and not on the PPG waveform. The method and devices propose predict the difference between heart rate variability values obtained with the PPG and the ECG using signal features extracted from the PPG signal only. Thus, it is possible to calculate heart rate variability metrics and their accuracy only from the PPG data, making it possible to use the method and devices in a prospective scenario. The additional advantage of using the heart rate variability error as quality is that no manual annotation of the PPG signal is involved, which reduces the risk of mistakes due to mislabeling. Moreover, the method and devices allow optimal selection of the threshold to use on the predicted heart rate variability error to distinguish between trustworthy and untrustworthy heart rate variability data points.

Summarizing and without excluding further possible embodiments, the following embodiments may be envisaged:

Embodiment 1: Computer implemented method for determining accuracy of heart rate variability comprising the following steps:
a) providing at least one photoplethysmogram obtained by at least one portable photoplethysmogram device;
b) Determining at least one signal feature by evaluating the photoplethysmogram;
c) Determining the accuracy of heart rate variability by using at least one trained model, wherein the signal features determined in step b) are used as input for the trained model.

Embodiment 2: The method according to the preceding embodiment, wherein the accuracy is used as quality indicator for heart rate variability data.

Embodiment 3: The method according to the preceding embodiment, wherein the accuracy is used for distinguishing between acceptable and non-acceptable heart rate variability data.

Embodiment 4: The method according to any one of the two preceding embodiments, wherein the method comprises comparing the accuracy to at least one threshold, wherein, if the accuracy is below the threshold, a heart rate variability data point is considered as acceptable, otherwise as non-acceptable.

Embodiment 5: The method according to the preceding embodiment, wherein the method comprises determining the at least one threshold.

Embodiment 6: The method according to any one of the preceding embodiments, wherein the photoplethysmogram comprises at least one signal, wherein the method comprises evaluating the signal, wherein the evaluation comprises one or more of interpolating the signal, resampling the signal, isolating signal component, analyzing considering non-overlapping windows, normalizing, identifying of peaks.

Embodiment 7: The method according to any one of the preceding embodiments, wherein the signal feature comprises at least one feature selected from the group consisting of: root mean square of successive differences (RMSSD), standard deviation of the R-to-R intervals (RRI) (SDNN), standard deviation of the RRIs in a current window, pnn50 from the photoplethysmogram (PPG), average heart rate from PPG in the current window, number of ectopic RRIs in the current window, minimum RRI value in the current window, variance of the RRIs in the current window, number of RRIs in the current window, 95th percentile of the RRIs in the current window, 5th percentile of the RRIs in the current window, variance of a raw PPG signal in the current window, max value of the raw PPG signal in the current window, min value of the raw PPG signal in the current window, average value of the raw PPG signal in the current window, standard deviation of the raw PPG signal in the current window, entropy of the raw PPG signal in the current window, kurtosis of the raw PPG signal in the current window, skewness of the raw PPG signal in the current window, variance of the filtered PPG signal in the current window, max value of the filtered PPG signal in the current window, min value of the filtered PPG signal in the current window, average value of the filtered PPG signal in the current window, standard deviation of the filtered PPG signal in the current window, kurtosis of the filtered PPG signal in the current window, skewness of the filtered PPG signal in the current window.

Embodiment 8: The method according to any one of the preceding embodiments, wherein the trained model comprises at least one model selected from the group consisting of: a linear regression model, e.g. comprising transformed features, such as log-transformed or polynomial; at least one non-linear Artificial Neural Network (ANN); at least one Support Vector Machine (SVM); at least one kernel based method; Tree regression; Random Forest.

Embodiment 9: The method according to any one of the preceding embodiments, wherein the method comprises at least one training step, wherein, in the training step, the trained model is trained on at least one training dataset, wherein the training dataset comprises a set of heart rate variability values determined by using at least one electrocardiogram device and heart rate variability values determined by using the photoplethysmogram device.

Embodiment 10: The method according to the preceding embodiment, wherein the method comprises determining at least one heart rate variability metric of the heart rate variability values determined by using at least one electrocardiogram device and determining at least one heart rate variability metric of the heart rate variability values determined by using the photoplethysmogram device, wherein the method comprises comparing the heart rate variability metrics against each other.

Embodiment 11: The method according to the preceding embodiment, wherein the method comprises determining at least one error of heart rate variability by combining the heart rate variability metric determined by using at least one electrocardiogram device and the heart rate variability metric of the heart rate variability values determined by using the photoplethysmogram device, wherein the error of heart rate variability is used together with signal features extracted from the photoplethysmogram for determining the trained model for determining the heart rate variability accuracy.

Embodiment 12: The method according to any one of the preceding embodiments, wherein the photoplethysmogram device comprises at least one illumination source and at least one photodetector.

Embodiment 13: A portable photoplethysmogram device, wherein the portable photoplethysmogram device is configured for determining accuracy of heart rate variability, wherein the portable photoplethysmogram device comprises at least one illumination source and at least one photodetector configured for determining at least one photoplethysmogram, the portable photoplethysmogram device further comprises at least one processing unit configured for determining at least one signal feature by evaluating the photoplethysmogram, wherein the processing unit is configured for determining the accuracy of heart rate variability by using at least one trained model, wherein the determined signal features are used as input for the trained model.

Embodiment 14: The portable photoplethysmogram device according to the preceding embodiment, wherein the portable photoplethysmogram device is configured for performing the method according to any one of the preceding embodiments.

Embodiment 15: A computer program comprising instructions which, when the program is executed by the portable photoplethysmogram device according to any one of the preceding embodiments referring to a portable photoplethysmogram device, cause the portable photoplethysmogram device to carry out steps a) to c) of the method according to any one of the preceding embodiments referring to a method.

Embodiment 16: A computer-readable storage medium comprising instructions which, when executed by the portable photoplethysmogram device according to any one of the preceding embodiments referring to a portable photoplethysmogram device, cause the portable photoplethysmogram device to carry out steps a) to c) of the method according to any one of the preceding embodiments referring to a method.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

### IN THE FIGURES

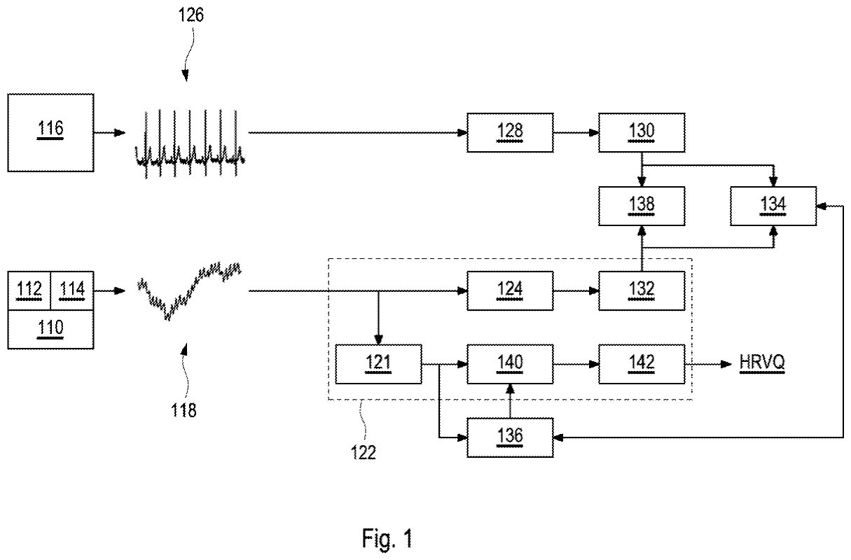

FIG. 1 shows a flow diagram of the method and at least one portable photoplethysmogram device according to the present invention;

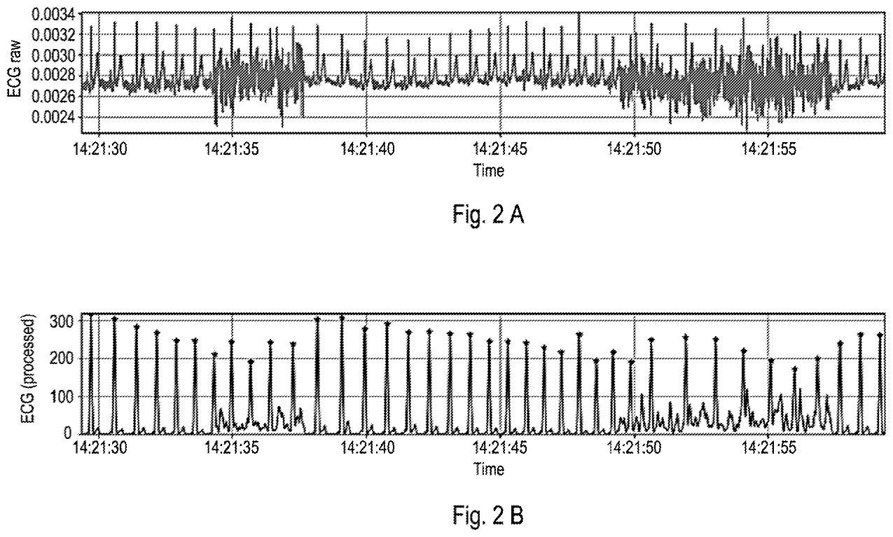

FIGS. 2A and 2B show an example of raw Electrocardiogram data and evaluated Electrocardiogram data;

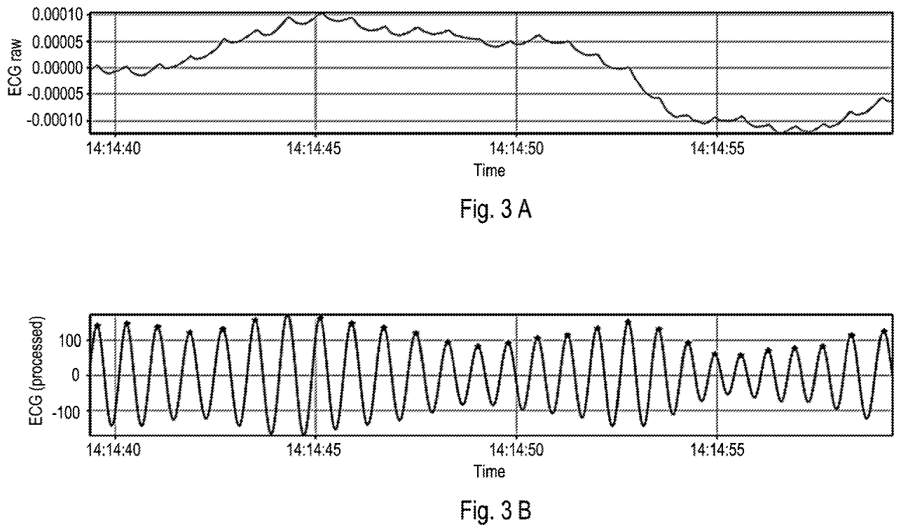

FIGS. 3A and 3B show an example of raw photoplethysmogram data and evaluated photoplethysmogram data;

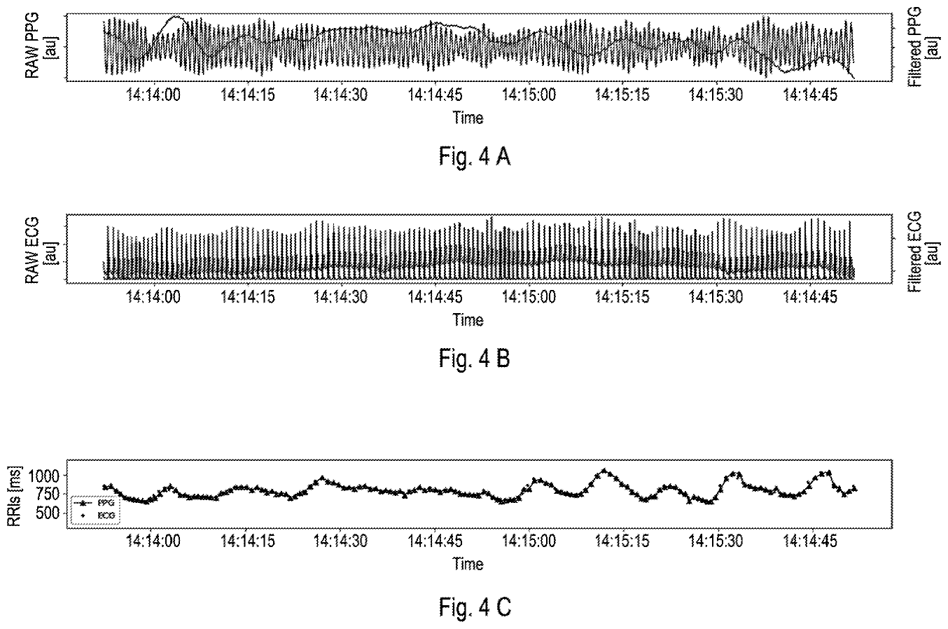

FIGS. 4A to 4C show an example of determining of R-to-R intervals;

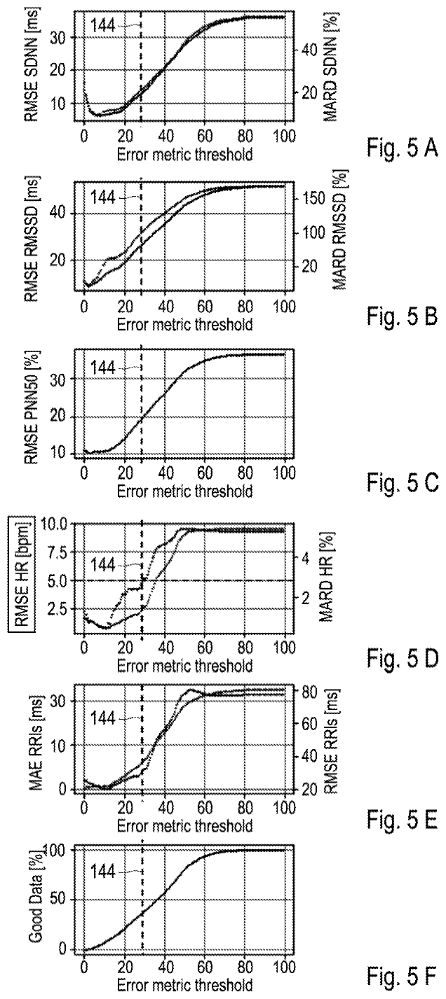

FIGS. 5A to 5F show heart rate variability accuracy results obtained for different threshold levels; and

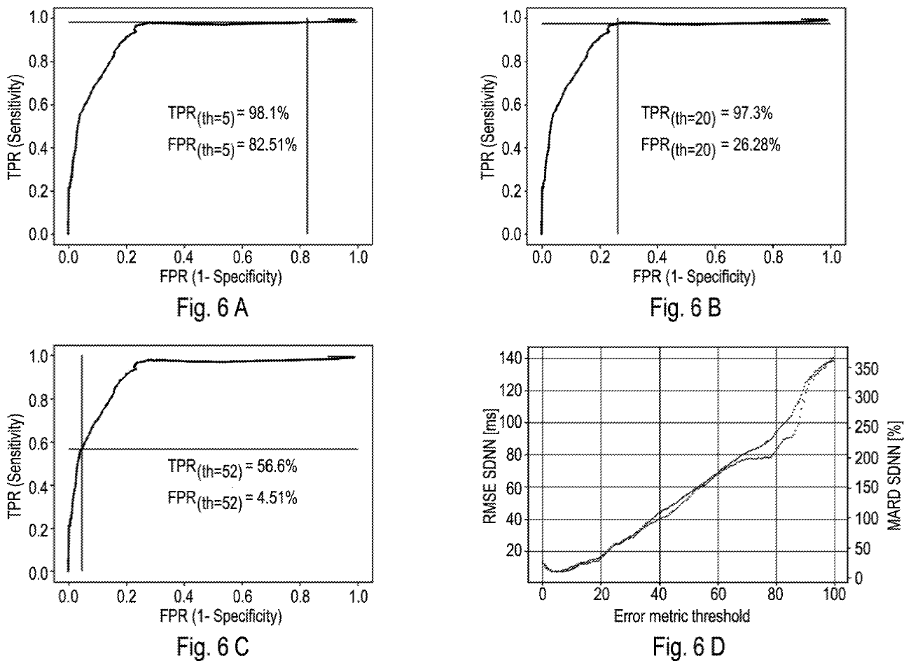

FIGS. 6A to 6D show in FIG. 6A to 6C True Positive Rate (TPR) and False Positive Rate (FPR) when binary classifying heart rate variability values depending on possible threshold levels and in FIG. 6D RMSE (left) and MARD (right) accuracy metrics for SDNN values considered as False Positives (FP) when a given value of the threshold is used on the estimated multivariate heart rate variability error metric to distinguish between accurate and inaccurate readings.

### DETAILED DESCRIPTION OF THE EMBODIMENTS

FIG. 1 shows a flow diagram of the method for determining accuracy of heart rate variability and at least one portable photoplethysmogram device 110 according to the present invention. The heart rate variability (HRV) may be a measure of regularity between consecutive heartbeats.

The photoplethysmogram device 110 is configured for determining at least one photoplethysmogram (PPG). The PPG may show development of a signal from the PPG device 110 over time.

The photoplethysmogram device 110 comprises at least one illumination source 112. The illumination source 112 may comprise at least one light source such as at least one light-emitting-diode (LED) transmitter. The illumination source 112 may be configured for generating at least one light beam for illuminating e.g. the skin on at least one part of the human body. The illumination source 112 may be configured for generating light in the red, infrared or green spectral region.

The photoplethysmogram device 110 may comprise at least one photodetector 114, in particular at least one photosensitive diode. The photodetector 114 may be configured for detecting a light beam, such as for detecting an illumination generated by at least one light beam. The photodetector 114 may be configured for detecting light from transmissive absorption and/or reflection in response to illumination by the light generated by the illumination source 112.

The PPG may comprise a plurality of beats. The heart rate variability may be measured by the variation in the beat-to-beat intervals, also denoted R-to-R intervals (RRI). Generally, an R wave is a section of an Electrocardiogram (ECG) signal consisting of a sharp raise followed by a sharp decrease of the signal. The morphology of a PPG signal may be different from the ECG one but still showing repetitive pattern due to heart beats. The heart rate variability may be defined as the variation of successive heartbeats.

The accuracy may be a measure for closeness of a measurement value to a certain value, in particular a true value. The true value may be a heart rate variability value determined using at least one ECG device 116.

The PPG device 110 may be wearable. For example, the PPG device 110 may be a wristwatch such as a smartwatch. Using a portable PPG device 110 may result in that disturbances can influence the HRV measurement such as motions artefacts. Uncontrolled conditions met in daily life may pose several challenges related to disturbances that can deteriorate a PPG signal 118 making the calculation of the HRV untrustworthy and not reliable.

The signal 118 may be at least one electronic signal of the PPG device 110, in particular of the photodetector 114, depending on detected light from transmissive absorption and/or reflection in response to illumination by the light generated by the illumination source 112.

The PPG device 110 may further comprise at least one processing unit 120 configured for determining at least one signal feature by evaluating the photoplethysmogram. The step of feature extraction is denoted with reference number 121. The photoplethysmogram may comprises at least one signal, also denoted as PPG signal 118. The evaluation of the PPG signal 118 may comprise one or more of interpolating the signal, resampling the signal, isolating signal components, analyzing considering non-overlapping windows, normalizing, identifying of peaks.

For example, the PPG signal 118 may be interpolated over a uniform time grid to account for slight fluctuations of sampling frequency, such as around 20 Hz. The PPG signal 118 may be resampled to increase the sampling frequency, such as to 1 kHz, for example by using an averaging filter of length 0.5 seconds and a Blackman window.

The PPG signal 118 may comprise a slow trend, often referred to DC component. Without being bound by this theory, this trend is likely due to respiration and other low frequency physiology-related modulations, see Julien, Claude. “The enigma of Mayer waves: facts and models.” Cardiovascular research 70.1 (2006): 12-21. The PPG signal 118 may comprise a pulsatile component, often referred to AC, due to blood volume variations synchronized with the heart beats. FIG. 4A shows a further example of a raw PPG signal 118 under rest conditions where the components are visible. To isolate the AC component from the PPG signal 118 a Morlet wavelet may be used, i.e.

a very selective band pass filter, centered around the frequency of interest, i.e. heart rate. For additional details about the Morlet wavelet reference is made toCohen, Michael X. “A better way to define and describe Morlet wavelets for time-frequency analysis.” NeuroImage 199 (2019): 81-86. It was found that the accuracy values are the key to make sure that the filtered signal contains the pulsatile component and not, for example, motion artefacts.

The PPG signal 118, in particular the resampled and interpolated PPG signal, may be analysed considering non-overlapping windows, such as windows of 20 seconds. For each window a median heart rate may be determined. The method may comprise using the median heart rate to build wavelet filter coefficients. Before applying the filter, a PPG waveform in a current window may be normalized by a DC mean value.

The peaks on the filtered PPG signal 118 may then be identified and/or determined and/or calculated looking at a combination of first and second derivatives of the signal. Identified peaks may then be concatenated until the last window that has been analyzed. A RRI time series, i.e. a specific number of consecutive peaks, may be filtered with a heuristic rule to make sure erroneous beats are excluded from the calculation of the HRV statistics. For example, a current RRI may be kept when it differs less than 30% from the previous one and the previous one, i.e. differs less than 30% from the one before, i.e. RIIᵢ₋₂. Otherwise the RRI may be removed from the RRI time series.

The signal features may comprise both statistics describing the PPG signal 118 as well as statistics describing the RRI distributions. The former ones may comprise one or more of variance, minimum, maximum, average, standard deviation, entropy, kurtosis and skewness values over raw and filtered PPG signals 118. The latter ones may comprise one or more of average RRIs and HR, the absolute number of filtered RRIs and the ratio between good and filtered RRIs, the minimum and maximum number of RIIs as well as the 5th and 95th RRI percentiles. The signal feature may be determined for a current time instant tᵢ considering RRIs temporally located between the current time instant tᵢ and a time instant tᵢ-wl, wherein wl is a window length ranging from seconds, e.g. 30 seconds, to minutes, such as 5 minutes. The signal feature comprise at least one feature selected from the group consisting of: root mean square of successive differences (RMSSD), standard deviation of the RRI intervals (SDNN), standard deviation of the RRIs in a current window, pnn50 from PPG, root mean square of pnn50, average RRI value from PPG in the current window, average heart rate from PPG in the current window, number of ectopic RRIs in the current window, ratio between number of ectopic and normal RRIs in the current window, minimum RRI value in the current window, maximum RRI value in the current window, variance of the RRIs in the current window, number of RRIs in the current window, 95th percentile of the RRIs in the current window, 5th percentile of the RRIs in the current window, variance of a raw, i.e. not filtered, PPG signal 118 in the current window, max value of the raw PPG signal 118 in the current window, min value of the raw PPG signal 118 in the current window, average value of the raw PPG signal 118 in the current window, standard deviation of the raw PPG signal in the current window, entropy of the raw PPG signal 118 in the current window, kurtosis of the raw PPG signal 118 in the current window, skewness of the raw PPG signal 118 in the current window, variance of the filtered PPG signal 118 in the current window, max value of the filtered PPG signal in the current window, min value of the filtered PPG signal in the current window, average value of the filtered PPG signal in the current window, standard deviation of the filtered PPG signal 118 in the current window, kurtosis of the filtered PPG signal 118 in the current window, skewness of the filtered PPG signal 118 in the current window. In the method according to the present invention all of these signal features may be determined or a subset of these signal features may be determined. It was found that the following subset of features is particular suitable for a reliable determination of accuracy of heart rate variability: root mean square of successive differences (RMSSD), standard deviation of the RRI intervals (SDNN), standard deviation of the RRIs in a current window, pnn50 from PPG, average heart rate from PPG in the current window, number of ectopic RRIs in the current window, minimum RRI value in the current window, variance of the RRIs in the current window, number of RRIs in the current window, 95^{th} percentile of the RRIs in the current window. The RMSSD may be determined by calculating the square root of the mean of the squares of the successive differences of consecutive RRIs:

$$RMSSD = \sqrt{\frac{{{Σ}_{i = 0}^{N - 1} ( {RRI}_{i} - {RRI}_{i + 1} )}^{2}}{N - 1}} .$$

The SDNN may be determined by calculating:

$$SDNN = \sqrt{\frac{{{Σ}_{i = 1}^{N} ( {RRI}_{i} - \overline{RRI} )}^{2}}{N - 1}} ,$$

where is RRI the average of the RRI in the considered time window. pnn50 is the proportion of NN50 divided by total number of RRIs, wherein NN50 is the number of pairs of successive RRIs that differ by more than 50 ms.

The processing unit 120 is configured for determining the accuracy of heart rate variability by using at least one trained model, wherein the determined signal features determined are used as input for the trained model. The steps shown inside box 122 of FIG. 1 may be performed by the processing unit 120.

The method may comprise at least one training step, wherein, in the training step, the trained model is trained on the at least one training dataset. The steps outside and inside the box 122 may be performed during the training step. The trained model may comprise at least one model selected from the group consisting of: a linear regression model, e.g. comprising transformed features, such as log-transformed or polynomial; at least one non-linear Artificial Neural Network (ANN), in particular at least one deep learning architecture such as Convolutional NN, Recurrent NN, Long Short Term Memory NN, and the like; at least one Support Vector Machine (SVM); at least one kernel based method; Tree regression; Random Forest.

The training dataset may comprise of a set of HRV values determined by using the ECG device 116 and HRV values determined by using the PPG device 110. ECG and PPG data may be collected simultaneously.

For example, for the experimental results shown in FIGS. 2 to 6, the training dataset consists of 20 recordings where ECG and PPG data are collected simultaneously from 20 healthy volunteers (4 female and 16 male) while performing a series of activities. The training dataset may be determined by performing at least one test protocol comprising the series of activities. During the test protocol, subjects were wearing a 3-LEDs ECG device 116 with sampling frequency at 1 kHz (BioRadio, by Neurotechnologies) and as PPG device 110 a smart watch on the wrist equipped with LEDs and photodiode for measuring PPG at 20 Hz (Samsung™ Gear Sport Smartwatch).

The protocol may comprise of a series of activities meant to induce HRV variations so to compare HRV over a wide range of values as well as inducing motion artefacts to test the ability of the algorithm and the quality metric to distinguish between accuracy and inaccurate HRV values. Some protocol activities, e.g. console gaming, mental stress manipulation and physical activity, may be included to reflect typical activities performed in daily life use of the PPG device. Pace breathing may be considered because it increases the range of HRV values through respiratory sinus arrhythmia, allowing the calculation of results over a broad range of variation and making easier the post alignment/synchronization of the time series obtained from the reference ECG and the PPG signals. The following table gives a list of an exemplary protocol:

| Activity | Duration |
|---|---|
| Screening & Informed consent process (while sitting, | — |
| at rest) | |
| Placement of ECG and PPG sensors (while sitting, | — |
| at rest) | |
| Baseline (sitting, at rest) | 5 minutes |
| Paced breathing (ladder of increasing respiratory | 5 minutes |
| frequencies from 5 to 20 breaths per minute with | |
| steps of 5) | |
| Console gameplay (PS4 Aaero) | 5 minutes |
| Orthostasis (standing, otherwise at rest) | 5 minutes |
| Mental stress manipulation (Serial 7s [subtraction | 5 minutes |
| by 7 from 700, with eyes closed, pronouncing aloud | |
| each response]) | |
| Physical activity manipulation (uninterrupted indoor | 5 minutes |
| walking along a pre-set circular path; same path for | |
| all subjects) | |
| Baseline (sitting, at rest) | 5 minutes |
| Retrieve PPG/ECG equipment and debrief | — |

The method may comprise analyzing ECG and PPG data to obtain the RRIs time series from which heart rate variability metrics can be derived. The method may comprise comparing the heart rate variability metrics against each other to obtain a measure of the accuracy.

The PPG signals may be evaluated as described above. The evaluation step is denoted with reference number 124. The signal features from the PPG signal and from the ECG signal may be calculated over the same time window. FIG. 2A shows an example of raw PPG data and evaluated, in FIG. 2B, with the proposed wavelet based algorithm to improve heart beats detection.

For the ECG data 126 comprising a plurality of ECG signals, a different evaluation may be performed. The raw ECG signal may be analyzed with a variation on the Pan-Tompkins algorithms, see Pan J, Tompkins W J. A real-time QRS detection algorithm. IEEE Trans Biomed Eng. 1985 March; 32(3):2. A Savitzky-Golay differentiation filter may be used to provide a filtered version of the raw signal first derivative, see Savitzky, A., Golay, M. J. E. “Smoothing and Differentiation of Data by Simplified Least Squares Procedures”. Analytical Chemistry. 1964, 36(8): 1627-39.doi:10.1021/ac60214a047. The ECG signal may be squared for emphasizing higher frequencies and filtered with a moving integrator filter, e.g. of width 60 ms, i.e. the average QRS complex width, for obtaining the ECG shape back with highlighted QRS complexes. The signal may be normalized with its envelope that is obtained at each time instant by filtering the root mean square of the signal in a rolling window of length Fs/2 with a Butterworth low pass filter with cutoff frequency at, e.g. 0.8 Hz, where Fs represents the sampling frequency of the ECG signal. Single heart beats may be identified on this normalized signal as the peaks exceeding a threshold that in our case was identified as the 90th percentile of the data in the current window. Each heart beat crossing the threshold may be subsequently checked manually to make sure no erroneous beat was included in the analysis. The evaluation of the ECG data 126 is shown with reference number 128 in FIG. 1. FIG. 3A shows an example of raw ECG data and evaluated, FIG. 3B, with the proposed algorithm to improve heart beats detection.

FIG. 4B shows a further example, of raw and filtered ECG signal with the Pan-Tompkins algorithm. FIG. 2A and FIG. 2B are combined onto FIG. 4B. The Figures refers to different time windows. In addition, in FIG. 4B the location of the peaks are not shown. Same for FIGS. 3A and 3B, that are combined into FIG. 4A. FIG. 4C shows a comparison of respective RRI intervals for a representative 2 minutes window for the PPG of FIG. 4A and the ECG of FIG. 4B.

The method may comprise determining at least one heart rate variability metric of the heart rate variability values determined by using at least one electrocardiogram device 116, denoted with reference number 130 in FIG. 1. The method may comprise determining at least one heart rate variability metric of the heart rate variability values determined by using the PPG device 110, denoted with reference number 132. The heart rate variability metric may be statistics calculated on RRIs contained inside a time window of specific length that can last from minutes to hours. For further details about the heart rate variability metric reference is made to Fei, Lu, et al, “Short- and long-term assessment of heart rate variability for risk stratification after acute myocardial infarction.” The American journal of cardiology 77.9 (1996): 681-684 and Mourot, Laurent, et al. “Short- and long-term effects of a single bout of exercise on heart rate variability: comparison between constant and interval training exercises.” European journal of applied physiology 92.4-5 (2004): 508-517. In particular, the heart rate variability metrics may be calculated on time window of minutes, what in the literature is sometimes referred as “short-term” heart rate variability. The heart rate variability metrics may be calculated in the specific 30, 60, 90, 120, 180, 240 and 300 seconds. Heart rate variability metrics may belong to different classes depending on the domain of the method used to analyze the RRIs.

For example, the heart rate variability metrics may comprise the time, frequency, non-linear and geometrical domains. The heart rate variability metrics may comprise the Root Mean of the Squared Differences (RMSSD) of consecutive RRIs:

$$RMSSD = \sqrt{\frac{{{Σ}_{i = 0}^{N - 1} ( {RRI}_{i} - {RRI}_{i + 1} )}^{2}}{N - 1}}$$

and the Standard Deviation of the NN intervals (SDNN):

$$SDNN = \sqrt{\frac{{{Σ}_{i = 1}^{N} ( {RRI}_{i} - \overline{RRI} )}^{2}}{N - 1}}$$

where is RRI the average of the RRI in the considered time window. Another metric derived from the interval differences may comprise the PNN50, that is, the number of consecutive RRIs differing more than 50 ms normalized by the total number of RRIs in the considered window.

The heart rate variability metrics obtained from the PPG and the ECG may be combined to define a heart rate variability error, denoted with reference number 134. The method may comprise determining at least one error of heart rate variability, i.e. the difference between heart rate variability values obtained with the PPG and the ECG. Specifically, for each heart rate variability metric an error may be determined, at each time instant i-th, as the absolute difference between the heart rate variability values obtained from the PPG and ECG signals:

Err_{SDNN.i}=|SDNN_{ECG,i}−SDNN_{PPG,i}|.

The method may comprise considering a combination of heart rate variability error metrics where at each time instant i-th, the multivariate error metric Err_{multivariate,i} is the average of the errors Err_{SDNN.i} for each heart rate variability metric.

The error of heart rate variability (HRVE) may be used together with signal features extracted from the PPG for determining the trained model for determining the heart rate variability accuracy itself, denoted with reference number 136. The method may comprise performing at least one multivariate supervised regression, wherein as input the at least one signal feature extracted from the photoplethysmogram may be used. The output may be the error between the heart rate variability metrics obtained from the PPG signal 118 and the ones obtained from the ECG signal.

For example, as model a linear model in the form may be used:

HRVE=Xβ

where HRVE is a (n×1) vector collecting the HRVEᵢ values Err_{multivariate.i}, X is the (n×p) matrix collecting the features obtained from the PPG and β is the (p×1) vector collecting the model coefficients. The i-th row of matrix X collects the p features calculated in the same time window of PPG data that is used to calculate the i-th heart rate variability value. For example, as estimation technique a Least Absolute Shrinkage and Selection Operator (LASSO) may be used. These techniques may comprise a L1 norm regularization and has the property of setting to zero coefficients in the model associated with unimportant features, allowing to control for complexity and avoiding overfitting, see Tibshirani R. Regression Shrinkage and Selection via the lasso. Journal of the Royal Statistical Society. Series B (methodological). 1996 58(1): 267-88.

In one embodiment the HRV accuracy may be trained with a subset of signal features:

$$H R {V}_{ACCURACY} ( {t}_{i} ) = {β}_{1} r m s s {d}_{p p g} ( {t}_{i} ) + {β}_{2} p n n 5 {0}_{p p g} ( {t}_{i} ) + {β}_{3} {avg\_hr}_{P P G} ( {t}_{i} ) + {β}_{4} n\_ectpc {\_rri}_{p p g} ( {t}_{i} ) + {β}_{5} min\_rri \_ppg ( {t}_{i} ) + + {β}_{6} {var}_{{rri}_{p p g} ( {t}_{i} )} + {β}_{7} {std}_{r r {i}_{p p g} ( {t}_{i} )} + {β}_{8} {n}_{{rri}_{p p g} ( {t}_{i} )} + {β}_{9} 95 {perc}_{r r {i}_{p p g} ( {t}_{i} )} ,$$

wherein βⱼ are the respective model coefficients, rmssd_ppg is the RMSSD from the PPG, pnn50_{ppg} is the pnn50 from the PPG, avg_hrp_{PPG} is the average heart rate from the PPG in the current window, n_ectpc_rri_{ppg} is the number of ectopic RRIs in the current window, min_rri_ppg is the minimum RRI value in the current window, var_rri_ppg is the variance of the RRIs in the current window, std_rri_ppg is the standard deviation of the RRIs in the current window, n_rri_ppg is the number of RRIs in the current window and 95perc_rri_ppg is the 95th percentile of the RRIs in the current window.

The method, in particular the training step, may comprise at least one validation step, wherein a Leave-One-Subject-Out Cross-Validation (LOSO-CV) is used. At each iteration N−1 subjects out of N subjects may be used to train the model. In the validation step, trained model is tested on the data from the subject that was left out from the training dataset, see Friedman, Jerome, Trevor Hastie, and Robert Tibshirani, The elements of statistical learning. Vol. 1. No. 10. New York: Springer series in statistics, 2001.

The trained model identified in step 136 can be used to estimate the heart rate variability error, e.g. prospectively, when ECG data is not available. This step is denoted with reference number 140 in FIG. 1.

The determined accuracy may be used as quality indicator for heart rate variability data. A better accuracy should be associated with a high quality and a low accuracy with a bad quality. The accuracy may be reflected by a quality metric. The heart rate variability determined from the photoplethysmogram may be an actual value in the quality metric. The quality metric may set a tolerance range that defines acceptable data points. The quality metric may be used for deciding and/or differentiating and/or distinguish between acceptable and non-acceptable heart rate variability data points.

The method may comprise comparing the accuracy to at least one threshold. If the accuracy is below the threshold, a heart rate variability data point may be considered as acceptable, otherwise as non-acceptable. The threshold may be used to distinguish between acceptable and unacceptable heart rate variability values. The method may comprise a binary decision to include or exclude a heart rate variability data point, denoted with reference number 142 in FIG. 1. The result of this decision is denoted as “HRVQ” in FIG. 1. The threshold may be a pre-determined or pre-defined threshold. The threshold may be set on the continuous HRVE values estimated by the trained model. When HRVE is below the threshold the respective heart rate variability value or data point may be considered with good quality and as “acceptable”, otherwise it is not and is considered as “non-acceptable”. An acceptable heart rate variability value may have a heart rate variability quality equal to 1 and a non-acceptable heart rate variability value may have a heart rate variability quality equal to 0.

The method may comprise determining the threshold, in particular at least one threshold level. Influences of different threshold levels may be tested as follows. For example, for all the considered heart rate variability metrics, the calculation of the heart rate variability accuracy may be performed using at least one performance metrics as a function of the threshold levels. Additionally or alternatively, the influences may be tested using an analysis considering errors arising from setting a threshold on a continuous value, HRVE, which is estimated by a model and thus presents uncertainty. The analysis may thus be highly dependent on the ability of the trained model to accurately predict HRVE. For example, a Receiver Operating Characteristic (ROC) analysis may be used using the true and the predicted values of HRVE for different threshold levels. For each threshold value, a confusion matrix may be calculated, a True Positive Rate (TPR), i.e. the rate of good quality HRV values classified as such, may be determined and a False Positive Rate (FPR), i.e. the number of inaccurate HRV values that are nevertheless included in the analysis because of the uncertainty in the predicted HRVE, may be determined. The heart rate variability accuracy of those points identified as FPR may have an indication of heart rate variability accuracy degradation derived from including these points.

FIGS. 5A to 5E show the HRV accuracy results for the multivariate error metric when the window length is 120 seconds obtained for different threshold levels, including the accuracy of pulse rate and the percentage of good data (relative to the total amount of data) included in the analysis as a function of the threshold. FIG. 5E shows the mean absolute error “MAE”. In general, the higher the threshold the more data is considered as accurate (see FIGS. 5F), but the accuracy of the HRV metrics decreases.

The vertical line 144, at threshold value around 30, is the accuracy used by FDA to clear a device for pulse rate monitoring as a medical device, see ANSI/AAMI EC13-1992, “Cardiac monitors, heart rate meters, and alarms”. This threshold gives an error in terms of RMSE for RMSSD around 30 ms and for SDNN around 15 ms. A threshold at 20 may be more desirable since the RMSE would drop below 15 ms for SDNN and around 20 for RMSSD.

Errors in the prediction of the HRV quality could cause the inclusions of data points that are actually inaccurate, as well as the exclusion of points that are accurate. To test what is the influence of these type of errors on the overall HRV accuracy the predicted and real values of HRVE were used to build the ROC curves in FIG. 6. FIGS. 6A to 6C show True Positive Rate (TPR) and False Positive Rate (FPR) when binary classifying HRV values as accurate or inaccurate depending on possible threshold levels and FIG. 6D shows RMSE (left) and MARD (right) accuracy metrics for SDNN values considered as False Positives (FP) when a given value of the threshold is used on the estimated multivariate HRV error metric (HRVQ) to distinguish between acceptable and non-acceptable readings. FIG. 6B shows that with a threshold at 20 returns a TPR of 97.1% but a FPR of 25.67%, meaning that a fourth of the point considered accurate should actually not be included because they are inaccurate. However, FIG. 6D, presenting the same analysis as in FIG. 5 but only for the FPR points, shows that FPR points still have a RMSE error for SDNN below 20 ms that, depending on the analysis, can still be considered acceptable.

The present invention proposes to defined a quality metric not on the PPG waveform but on the HRV metrics, which is associated with the HRV accuracy. A higher HRV accuracy, lower HRV error, is associated with a better quality. Using the ECG signal to calculate HRV metrics that are considered reliable, avoids the problem of manually annotating the PPG signal 118, a tedious, subjective, process that could potentially results in erroneous labelling and misleading results. While the waves in the ECG signal are often clearly visible, these can be labelled with relative safety, the PPG waves are usually more complicated to assess. A quality measure based on a combination of several HRV metrics errors is more robust than a quality measure based on a individual HRV metric error. This quality is thus universal, in the sense that can be used for all HRV metrics and there is no need to estimate a quality for each individual metric. The more accurate the prediction of the HRV error, the lower the FPR error will be.

### LIST OF REFERENCE NUMBERS

110 portable photoplethysmogram device
112 illumination source
114 photodetector
116 ECG device
118 PPG signal
120 processing unit
121 feature extraction
122 box
124 evaluation step
126 ECG data
128 evaluation of the ECG data
130 determining at least one heart rate variability metric
132 determining at least one heart rate variability metric
134 Determining of heart rate variability error
136 determining the heart rate variability accuracy
138 determining at least one performance metrics
140 estimate heart rate variability error
142 binary decision
144 vertical line

1. A computer implemented method for determining accuracy of heart rate variability comprising the following steps:
a) providing at least one photoplethysmogram obtained by at least one portable photoplethysmogram device;
b) determining at least one signal feature by evaluating the photoplethysmogram;
c) determining the accuracy of heart rate variability by using at least one trained model, wherein the signal features determined in step b) are used as input for the trained model;
wherein the accuracy is used for distinguishing between acceptable and non-acceptable heart rate variability data, wherein the method comprises comparing the accuracy to at least one threshold, wherein, if the accuracy is below the threshold, a heart rate variability data point is considered as acceptable, otherwise as non-acceptable.

2. The method according to claim 1, wherein the accuracy is used as quality indicator for heart rate variability data.

3.-4. (canceled)

5. The method according to claim 1, wherein the method comprises determining the at least one threshold.

6. The method according to claim 1, wherein the photoplethysmogram comprises at least one signal, wherein the method comprises evaluating the signal, wherein the evaluation comprises one or more of interpolating the signal, resampling the signal, isolating signal component, analyzing considering non-overlapping windows, normalizing, identifying of peaks.

7. The method according to claim 1, wherein the signal feature comprises at least one feature selected from the group consisting of: root mean square of successive differences (RMSSD), standard deviation of the R-to-R intervals (RRI) (SDNN), standard deviation of the RRIs in a current window, pnn50 from the photoplethysmogram (PPG), average heart rate from PPG in the current window, number of ectopic RRIs in the current window, minimum RRI value in the current window, variance of the RRIs in the current window, number of RRIs in the current window, 95^{th} percentile of the RRIs in the current window, 5th percentile of the RRIs in the current window, variance of a raw PPG signal in the current window, max value of the raw PPG signal in the current window, min value of the raw PPG signal in the current window, average value of the raw PPG signal in the current window, standard deviation of the raw PPG signal in the current window, entropy of the raw PPG signal in the current window, kurtosis of the raw PPG signal in the current window, skewness of the raw PPG signal in the current window, variance of the filtered PPG signal in the current window, max value of the filtered PPG signal in the current window, min value of the filtered PPG signal in the current window, average value of the filtered PPG signal in the current window, standard deviation of the filtered PPG signal in the current window, kurtosis of the filtered PPG signal in the current window, skewness of the filtered PPG signal in the current window.

8. The method according to claim 1, wherein the trained model comprises at least one model selected from the group consisting of: a linear regression model, e.g. comprising transformed features, such as log-transformed or polynomial; at least one non-linear Artificial Neural Network (ANN); at least one Support Vector Machine (SVM); at least one kernel based method; Tree regression; Random Forest.

9. The method according to claim 1, wherein the method comprises at least one training step, wherein, in the training step, the trained model is trained on at least one training dataset, wherein the training dataset comprises a set of heart rate variability values determined by using at least one electrocardiogram device and heart rate variability values determined by using the photoplethysmogram device.

10. The method according to claim 9, wherein the method comprises determining at least one heart rate variability metric of the heart rate variability values determined by using at least one electrocardiogram device and determining at least one heart rate variability metric of the heart rate variability values determined by using the photoplethysmogram device, wherein the method comprises comparing the heart rate variability metrics against each other.

11. The method according to claim 10, wherein the method comprises determining at least one error of heart rate variability by combining the heart rate variability metric determined by using at least one electrocardiogram device and the heart rate variability metric of the heart rate variability values determined by using the photoplethysmogram device, wherein the error of heart rate variability is used together with signal features extracted from the photoplethysmogram for determining the trained model for determining the heart rate variability accuracy.

12. The method according to claim 1, wherein the photoplethysmogram device comprises at least one illumination source and at least one photodetector.

13. A portable photoplethysmogram device, wherein the portable photoplethysmogram device is configured for determining accuracy of heart rate variability, wherein the portable photoplethysmogram device comprises at least one illumination source and at least one photodetector configured for determining at least one photoplethysmogram, the portable photoplethysmogram device further comprises at least one processing unit configured for determining at least one signal feature by evaluating the photoplethysmogram, wherein the processing unit is configured for determining the accuracy of heart rate variability by using at least one trained model, wherein the determined signal features are used as input for the trained model, wherein the accuracy is used for distinguishing between acceptable and non-acceptable heart rate variability data, wherein the portable photoplethysmogram device is configured for comparing the accuracy to at least one threshold, wherein, if the accuracy is below the threshold, a heart rate variability data point is considered as acceptable, otherwise as non-acceptable.

14. The portable photoplethysmogram device according to claim 13, wherein the portable photoplethysmogram device is configured for performing the method according to claim 1.

15. A computer program comprising instructions which, when the program is executed by the portable photoplethysmogram device according to claim 13 referring to a portable photoplethysmogram device, cause the portable photoplethysmogram device to carry out steps a) to c) of the method according to claim 1.

16. A computer-readable storage medium comprising instructions which, when executed by the portable photoplethysmogram device according to claim 13 referring to a portable photoplethysmogram device, cause the portable photoplethysmogram device to carry out steps a) to c) of the method according to claim 1.
